(19) United States
(12) Patent Application Publication
(10) Pub. No.: US 2008/0181946 A1
(43) Pub. Date: Jul. 31, 2008

(54) Controlled Release Delivery System For Metformin

(76) Inventors: Braj Bhushan Lohray, Gujarat (IN); Sandip B. Tiwari, Gujarat (IN)

Correspondence Address: NIXON & VANDERHYE, PC, 901 NORTH GLEBE ROAD, 11TH FLOOR, ARLINGTON, VA, 22203, US

(21) Appl. No.: 11/596,371

(22) PCT Filed: May 5, 2005
(86) PCT No.: PCT/IN05/00148

§ 371 Date: Nov. 13, 2007

(30) Foreign Application Priority Data
May 14, 2004 (IN) 557/MUM/2004

(51) Int. Cl. A61K 9/36; A61K 31/155; A61K 31/4439; A61P 7/00; A61K 31/402

(52) U.S. Cl. 424/465; 514/632; 514/342; 514/423

(57) ABSTRACT

The invention relates to a controlled release delivery system for Metformin comprising of (a) therapeutically effective amount of Metformin or pharmaceutically acceptable salts there of; (b) hydrophilic polymers and (c) hydrophobic lubricating agent(s). The invention also relates to a method of making the said solid dosage form.

### FIELD OF INVENTION

The invention relates to a controlled release delivery system for Metformin comprising of (a) therapeutically effective amount of Metformin or pharmaceutically acceptable salts there of; (b) hydrophilic polymers and (c) hydrophobic lubricating agent(s). The invention also relates to a method of making the said solid dosage form.

### BACKGROUND OF INVENTION

Diabetes mellitus of type II is a progressive metabolic disorder with diverse pathologic manifestations and is often associated with lipid metabolism and glycometabolic disorders. The long-term effects of diabetes result from its vascular complications; the microvascular complications of retinopathy, neuropathy and nephropathy and the macrovascular complications of cardiovascular, cerebrovascular and peripheral vascular diseases. Initially, diet and exercise is the mainstay of treatment of type II diabetes. However, these are followed by administration of oral hypoglycemic agents. Current drugs used for managing type II diabetes and its precursor syndromes such as insulin resistance, include classes of compounds, such as, among others, biguanides, thiazolidinediones and sulfonylureas.

Biguanides, represented principally by metformin hydrochloride, phenformin and buformin, help in the control of blood glucose by inhibiting hepatic glucose production, reducing intestinal absorption of glucose and enhancing peripheral glucose uptake. Biguanides, especially metformin hydrochloride, lowers both basal and post-prandial plasma glucose and thus improves tolerance of glucose in patients. Metformin hydrochloride exerts normoglycemic action with reduced risk of lactic acidosis and is also known to lower blood triglyceride levels. It is therefore a preferred mode of therapy among biguanides.

Metformin hydrochloride has intrinsically poor permeability in the lower portion of the GIT leading to absorption almost exclusively in the upper part of GIT. Its oral bioavailability is in the range of 40 to 60% decreasing with increasing dosage which suggests some kind of saturable absorption process, or permeability limited absorption. It also has a very high water solubility (>300 mg/ml at 25° C.). This is the challenge or difficulty in providing the active pharmaceutical agent at slow rate and also controlling the initial burst effect from the dosage unit. These difficulties are further compounded by high unit doses of 500-mg, 750-mg and 1000-mg usually required for Metformin hydrochloride.

The gastrointestinal tract functions to propel ingested material from the alimentary canal and the absorption is maximum in the upper part of GI tract, where as in the large intestine water is absorbed/secreted as part of body fluid regulation.

In the case of metformin hydrochloride, it is desirable to provide a dosage form that allows extended delivery of the drug and has a prolonged gastric residence via swelling of the system rather than unfolding or expanding of a folded device, and that may be manufactured on a commercial scale. The prolonged gastric residence time is required due to the window of absorption seen with metformin hydrochloride. Another problem for extended delivery of metformin hydrochloride is its very high water solubility. High levels of polymer would be needed if one desires to provide controlled release of the drug. More over, the use of hydrophilic polymers alone for controlled drug release could result in a rapid and variable initial release (burst) of drug from an extended release dosage form. This thus may give rise to difficulty in providing a true control of drug release and minimal inter-patient variability in drug plasma levels (arising from the possibility of variable burst of drug from tablets given to different patients).

There are number of different modified release dosage forms available commercially. However, some of these are expensive to manufacture and can be difficult to swallow, particularly in elderly patients. However since many modified release dosage forms contain comparatively large amounts of active ingredient it is often necessary to include large amounts of suitable excipients to achieve appropriate controlled release profiles which results in increase in the size of the dosage form.

Some of the techniques to make modified release dosage form of drugs as described in prior art are as follows:

WO99/47128 discloses a method of prolonging the release of a highly water-soluble drug. A biphasic controlled release delivery system for metformin hydrochloride, which has prolonged gastric residence and that swells following hydration is described.

WO 02/28181 describes a monolithic sustained release formulation of metformin hydrochloride. The method of making the formulation involves hot melt granulation followed by wet granulation with binders of extrusion.

WO 2004/012699 A2 discloses modified release dosage form comprising of a highly soluble active ingredient, which utilizes dual retard technique comprising micro matrix particles containing active ingredient(s) and one or more hydrophobic release controlling agents and coating of one or more hydrophobic release controling agents.

US2003/0170302 discloses extended release pharmaceutical tablet of metformin comprising a core containing metformin and a coating permeable to metformin. U.S. Pat. No. 6,475,521 describes biphasic controlled release delivery system for high solubility pharmaceutical using biphasic controlled release delivery system for treating diabetes.

US application US2004/0086566 describes a waxy matrix dosage forms comprising a pharmaceutically effective amount of metformin and a waxy matrix material. This publication relies on the use of waxy matrix for the preparation of tablets or other dosage forms. Here, the process of melting requires use of special equipment and the process is quite tedious. It is in a way expensive also. In brief one can say, the process is tedious, expensive and time consuming as the manufacture of such dosage forms requires special equipment, time for heating and cooling the mass, difficulty in sieving the molten mass and higher wear and tear associated with the process. Here, the use of high temperature melting devices is associated with the risk of accidental injury or burns.

Hydrophobic matrix systems have technical difficulties in terms of production and product performance. Where as hydrophilic matrix systems are technically easy to manufacture and have desirable pharmacotechnical properties. The difference is of technology and simplicity in terms of industrially applicable and feasible, as with hot melt technique or using hydrophobic polymers, reproducibility is difficult & there is batch to batch variation as well as within the batch variation.

These systems generally can provide for modified release for selected active ingredients with low dose or low water solubility. However, when a highly soluble or high dose active ingredient is used, most of these systems have the disadvantages such as comparatively high payload of inactive ingredient(s) thus making dosage form bulky and expensive or lead to burst effect wherein the active drug may be released prematurely without providing prolonged drug release profile. Also some of the aforementioned delivery systems requires special manufacturing setup, are expensive in terms of processing conditions, operational time and simplicity.

It is in light of this extensive background that the applicants entered their study in an attempt to find out a new dosage form for highly water soluble pharmaceuticals such as metformin hydrochloride, which provide for prolonged release of the drug so that the dosing regimen of pharmaceutically effective amount of metformin hydrochloride once daily, may be achieved while providing effective control of plasma glucose. The delivery system manufactured using the present invention is operationally simple, easy to manufacture, cost effective with high turn over rates

The delivery system of the present invention can be manufactured using the conventional processing equipment generally used in the manufacture of the pharmaceutical dosage forms.

### PREFERRED EMBODIMENTS OF THE INVENTION

In an embodiment of the present invention is provided a controlled release drug delivery system for metformin comprising of: (a) pharmaceutically effective amount of Metformin or its pharmaceutically acceptable salts, particularly Metformin hydrochloride (b) hydrophilic polymers and (c) hydrophobic lubricating agent(s).

Another embodiment of the present invention provides a process for preparation of controlled drug delivery system of Metformin or its pharmaceutically acceptable salts, particularly Metformin hydrochloride.

A further embodiment of the present invention discloses a dosage form, which gives accurate dosing and is operationally simple to manufacture at a large scale.

A still further embodiment of the present invention provides a controlled release drug delivery system for Metformin hydrochloride for controlled delivery with improved patient compliance on account of once daily administration and ease of administration.

### DESCRIPTION OF THE INVENTION

Accordingly the present invention provides a controlled release drug delivery system for Metformin or its pharmaceutically acceptable salts, preferably metformin hydrochloride. The present invention also dislcoses the method of making the said solid dosage form of Metformin or its pharmaceutically acceptable salts, preferably metformin hydrochloride.

According to the present invention, a solid dosage form of Metformin is provided comprising: (a) pharmaceutically effective amount of Metformin or pharmaceutically acceptable salts thereof (b) suitable hydrophilic polymers and (c) suitable hydrophobic lubricating agent(s). The unit dosage form may optionally comprise other pharmaceutical processing aids.

The examples of hydrophillic polymers includes but are not limited to cellulosic derivatives such as hydroxypropyl methylcellulose, hydroxypropyl cellulose, sodium carboxy methylcellulose, carboxypolymethylene, carboxymethylcellulose calcium, carboxymethylcellulose sodium, hydroxyethyl cellulose, sodium alginate, xantham gum, or mixtures thereof.

Examples of hydrophobic lubricating agents includes but are not limited to pharmaceutically acceptable hydrogenated vegetable oils of Type I such as hydrogenated castor oil, hydrogenated cottonseed oil, hydrogenated palm oil, hydrogenated soyabean oil and the like or mixture thereof, fatty acid esters such as glyceryl monostearate, glyceryl diastearate, glycerol monooleate, glyceryl behenate, gylceryl palmitostearate, light mineral oil and the like or mixtures thereof.

The examples of other pharmaceutical aids includes commonly used pharmaceutical aids such as binder, diluent and the like. Examples of binders include Povidone, hydroxypropylcellulose and the like. Diluents include microcrystalline cellulose, lactose, dicalcium phosphate and the like.

According to the present invention, the process of preparation of a controlled release drug delivery system for metformin comprises steps of:
a. granulating metformin with suitable hydrophilic polymers in a suitable solvent such as water and/or organic solvent such as isopropyl alcohol, ethyl alcohol, methylene chloride, acetone or mixture there of, optionally using other pharmaceutical aids.
b. drying and sizing the obtained granules.
c. lubricating the granules of step (b) with hydrophobic lubricating agents and compressing into a suitable dosage form.

In another embodiment of the present invention, some portion of the hydrophilic polymers can be added extragranularly along with hydrophobic lubricants and compressing the blend to obtain the suitable dosage form.

The present invention also relates to pharmaceutical composition of Metformin or its pharmaceutically acceptable salts, particularly Metformin hydrochloride prepared according to the present invention.

The present invention relates to the use of the pharmaceutical composition of Metformin hydrochloride prepared according to the present invention for the treatment of diabetes and other related disorders.

In another embodiment of the present invention, the formulation of Metformin prepared according to the present invention is formulated in a bilayer tablet wherein the second layer may comprise of other oral hypoglycemic agent or insulin sensitiser or secretogouge along with metformin.

### Advantages of the Present Invention:

1. The controlled drug delivery system of the present invention results in a convenient dosage form with better patient compliance.
2. The controlled drug delivery system of the present invention provides the controlled release of Metformin in a pH independent manner.
3. The operational simplicity and cost effectiveness of the present invention makes it suitable for industrial application.
4. Hydrophobic matrix systems have technical difficulties in terms of production and product performance, whereas hydrophilic matrix systems are technically easy to manufacture and have desirable pharmacotechnical properties.

The process described in the present invention is demonstrated in the examples given below. These examples are provided as illustration and should not be considered as limiting the scope of the invention in any way.

### EXAMPLE 1

The formulation of Metformin was prepared according to the following formula:

| Ingredient | % |
|---|---|
| Metformin hydrochloride | 47.62 |
| Hydroxypropyl methyl cellulose | 38.10 |
| (Methocel K15M) | |
| Microcrystalline cellulose | 4.86 |
| (Avicel PH 101) | |
| Povidone K 90 | 6.29 |
| Glyceryl behenate | 3.14 |
| (Compritol 888 ATO) | |
| Isopropyl alcohol:water (10:90 v/v) | q.s |

### Process:

Metformin hydrochloride, Hydroxyproryl methylcellulose, Microcrystalline cellulose and Povidone were mixed in a double-cone blender & then granulated with Isopropyl alcohol:water (10:90) in a granulator. The wet mass was sifted through #10 mesh (ASTM) & dried the granules to get a moisture content of less than 2%. The dried granules were sifted through #20 mesh (ASTM) and mixed with Glyceryl behanate.

The dissolution profile of the tablets was as follows:

Medium: Demineralised water (900 mL)

Method: USP Apparatus Type I (100 RPM).

| Time | |
|---|---|
| (hr) | % drug released |
| 1 | 31.72 |
| 2 | 46.51 |
| 4 | 65.06 |
| 6 | 80.68 |
| 8 | 90.47 |
| 10 | 98.88 |
| 12 | 98.33 |
| 14 | 101.59 |

### EXAMPLE 2

Example 2 was reproduced according to the same manufacturing process described above in example 1, with the following composition in the formulation:

| Ingredient | % |
|---|---|
| Metformin hydrochloride | 47.62 |
| Hydroxypropyl methyl cellulose | 38.10 |
| (Methocel K15M) | |
| Microcrystalline cellulose | 4.86 |
| (Avicel PH 101) | |
| Povidone K 90 | 6.29 |
| Glyceryl behenate | 3.14 |
| (Compritol 888 ATO) | |
| Isopropyl alcohol:water (70:30 v/v) | q.s |

Dissolution profile: The dissolution of the above formulation was carried out in a manner similar to that described in example 1 and the release profile was:

| Time | |
|---|---|
| (hr) | % drug released |
| 1 | 29.22 |
| 2 | 43.33 |
| 4 | 61.21 |
| 6 | 75.29 |
| 8 | 85.85 |
| 10 | 93.81 |
| 12 | 98.33 |
| 14 | 99.89 |

### EXAMPLE 3

Example 3 was reproduced according to the same manufacturing process described above in example 1, with the following composition in the formulation:

| Ingredient | % |
|---|---|
| Metformin hydrochloride | 47.62 |
| Hydroxypropyl methyl cellulose | 40.00 |
| (Methocel K15M) | |
| Microcrystalline cellulose | 3.38 |
| (Avicel PH 101) | |
| Povidone | 6.00 |
| Glyceryl behenate | 3.00 |
| (Compritol 888 ATO) | |
| Isopropyl alcohol:water (70:30 v/v) | q.s |

Dissolution profile: The dissolution of the above formulation was carried out by a similar process as described in example 1 and the release profile was:

| Time | |
|---|---|
| (hr) | % drug released |
| 1 | 32.65 |
| 2 | 47.10 |
| 4 | 65.41 |
| 6 | 80.08 |
| 8 | 88.67 |
| 10 | 93.90 |
| 12 | 97.55 |
| 14 | 97.96 |

### EXAMPLE 4

Example 4 was produced according to the manufacturing process described below with following composition:

| Ingredient | % |
|---|---|
| Metformin hydrochloride | 66.37 |
| Hydroxypropyl methyl cellulose | 8.84 |
| (Methocel K100M) | |
| Microcrystalline cellulose | 8.84 |
| (Avicel PH 101) | |
| Hydroxypropyl methyl cellulose | 13.27 |
| (Methocel K100M) | |
| Glyceryl behenate | 2.65 |
| (Compritol 888 ATO) | |
| Isopropyl alcohol:water (40:60 v/v) | q.s |

Metformin hydrochloride, hydroxypropyl methylcellulose (8.84%) and microcrystalline cellulose were mixed in a double-cone blender and then granulated with Isopropyl alcohol:water (40:60 v/v) in a granulator. The wet mass was sifted through sieve #10 (ASTM) and dried. The dried granules were sifted through sieve #20 (ASTM) and mixed with hydroxypropyl methylcellulose (13.27%) and glyceryl behenate. The granules were then compressed into tablets using rotary compression machine.

Dissolution profile: The dissolution of the above formulation was carried out by a similar process as described in example 1 and the release profile was:

| Time | |
|---|---|
| (hr) | % drug released |
| 1 | 34.36 |
| 2 | 51.58 |
| 4 | 72.2 |
| 6 | 85.84 |
| 8 | 94.65 |
| 10 | 98.54 |
| 12 | 98.96 |
| 14 | 99.41 |

### EXAMPLE 5

Example 5 was reproduced according to the same manufacturing process described above in example 4, except the change that the quantity of the extragranular hydroxypropyl methylcellulose was 10.62%.

| Ingredient | % |
|---|---|
| Metformin hydrochloride | 66.37 |
| Hydroxypropyl methyl cellulose | 8.85 |
| (Methocel K100M) | |
| Microcrystalline cellulose | 8.85 |
| (Avicel PH 101) | |
| Povidone K 90 | 2.65 |
| Hydroxypropyl methyl cellulose | 10.62 |
| (Methocel K100M) | |
| Glyceryl behenate | 2.65 |
| (Compritol 888 ATO) | |
| Isopropyl alcohol:water (40:60 v/v) | q.s |

Dissolution profile: The dissolution of the above formulation was carried out by a similar method as described in example 1 and the release profile was:

| Time | |
|---|---|
| (hr) | % drug released |
| 1 | 33.62 |
| 2 | 49.74 |
| 4 | 70.25 |
| 6 | 83.68 |
| 8 | 92.47 |
| 10 | 96.63 |
| 12 | 98.22 |
| 14 | 98.47 |

### EXAMPLE 6

Example 6 was reproduced according to the same manufacturing process described above in example 4 with the following composition:

| Ingredient | % |
|---|---|
| Metformin hydrochloride | 66.37 |
| Hydroxypropyl methylcellulose (Methocel | 8.85 |
| K100 M) | |
| Microcrystalline cellulose (Avicel PH 101) | 8.85 |
| Povidone (K-90) | 2.65 |
| Hydroxypropyl methylcellulose (Methocel | 10.62 |
| K100 M) | |
| Glyceryl behenate | 2.65 |
| Isopropyl alcohol:water (40:60 v/v) | q.s |

The tablets were spray coated with 4% w/v hydroxypropyl methylcellulose (6 cps) in 50:50 v/v isopropyl alcohol:methylene chloride solution containing standard coating aids (Titanium dioxide, talc and polyethylene glycol) to a weight gain of 3% of total tablet weight.

Dissolution profile: The dissolution of the above formulation was carried out by a similar process as described in example 1 and the release profile was:

| Time | |
|---|---|
| (hr) | % drug released |
| 1 | 28.2 |
| 2 | 47.40 |
| 4 | 67.51 |
| 6 | 78.82 |
| 8 | 89.75 |
| 10 | 93.63 |
| 12 | 93.20 |
| 14 | 98.47 |

### EXAMPLE 7

Example 7 was reproduced according to the same manufacturing process described above in example 1, with the following composition in the formulation:

| Ingredient | % |
|---|---|
| Metformin hydrochloride | 46.94 |
| Pioglitazone hydrochloride | 1.4 |
| Hydroxypropyl methylcellulose | 37.55 |
| (Methocel K15M) | |
| Microcrystalline cellulose (Avicel | 4.83 |
| PH101) | |
| Povidone (K-90) | 6.19 |
| Glyceryl behenate | 3.09 |
| Isopropyl alcohol:water (70:30 v/v) | q.s. |

### EXAMPLE 8

Example 8 was reproduced according to the same manufacturing process described above in example 1, with the following composition in the formulation:

| Ingredient | % |
|---|---|
| Metformin hydrochloride | 47.52 |
| Glimepiride | 0.19 |
| Hydroxypropyl methylcellulose | 38.02 |
| (Methocel K15M) | |
| Microcrystalline cellulose (Avicel | 4.86 |
| PH101) | |
| Povidone (K-90) | 6.27 |
| Glyceryl behenate | 3.13 |
| Isopropyl alcohol:water (70:30 v/v) | q.s. |

### EXAMPLE 9

Example 6 is reproduced according to the same manufacturing process described above in example 4.

| Ingredient | % |
|---|---|
| Metformin hydrochloride | 66.37 |
| Hydroxypropyl methylcellulose (Methocel | 8.85 |
| K100 M) | |
| Microcrystalline cellulose (Avicel PH 101) | 8.85 |
| Povidone (K-90) | 2.65 |
| Hydroxypropyl methylcellulose (Methocel | 10.62 |
| K100 M) | |
| Glyceryl Palmitostearate (Precirol ATO 5) | 2.65 |
| Isopropyl alcohol:water (40:60 v/v) | q.s |

1. A controlled release delivery system for Metformin which comprises:
(a) a therapeutically effective amount of Metformin or its pharmaceutically acceptable salts,
(b) hydrophilic polymer(s) and,
(c) hydrophobic lubricating agent(s) selected from hydrogenated castor oil, hydrogenated cottonseed oil, hydrogenated palm oil, hydrogenated soybean oil, glyceryl monostearate, glyceryl diastearate, glycerol monooleate, glyceryl behenate, gylceryl palmitostearate, light mineral oil or combinations thereof.

2. A controlled drug delivery system according to claim 1 comprising, hydrophilic polymer(s) selected from hydroxypropyl methylcellulose, hydroxypropyl cellulose, sodium carboxymethylcellulose, carboxypolymethylene, carboxymethylcellulose calcium, carboxymethylcellulose sodium, hydroxyethyl cellulose, sodium alginate, xantham gum, or combinations thereof.

3. A controlled release delivery system for Metformin which comprises:
a) a therapeutically effective amount of Metformin or its pharmaceutically acceptable salts,
b) hydrophilic polymer(s) selected from hydroxypropyl methylcellulose, hydroxypropyl cellulose, sodium caroboxymethylcellulose, carboxypolymethylene, carboxymethylcellulose calcium, carboxymethylcellulose sodium, hydroxyethyl cellulose, sodium alginate, xantham gum, or combinations thereof and,
c) hydrophobic lubricating agent(s) selected from hydrogenated castor oil, hydrogenated cottonseed oil, hydrogenated palm oil, hydrogenated soybean oil, glyceryl monostearate, glyceryl diastearate, glycerol monooleate, glyceryl behenate, gylceryl palmitostearate, light mineral oil or combinations thereof.

4. A controlled release delivery system according to claim 1, comprising pharmaceutically acceptable hydrophilic polymers, on a weight basis, about 5% to 95%, preferably present in an amount from about 10% to about 75%, more preferably from about 15% to about 55%.

5. A controlled release delivery system according to claim 1, comprising pharmaceutically acceptable hydrophobic lubricating agent(s) on a weight basis, about 0.5% to about 15%, preferably present in an amount from about 1% to about 10%, more preferably from about 1.5% to about 7.5% by weight.

6. A controlled release delivery system for metformin according to claim 1, wherein the delivery system further comprises of additional oral hypoglycemic agent selected from glyburide, glimepiride, pioglitazone, rosiglitazone, nateglinide and repaglinide.

7. A controlled release delivery system for Metformin which comprises:
a) a therapeutically effective amount of Metformin or its pharmaceutically acceptable salts,
b) hydrophilic polymer(s),
c) hydrophobic lubricating agent(s).
d) optionally, coating the tablet using coating agents selected from hydroxypropylcellulose, hydroxypropylcellulose, hydroxyethylmethylcellulose, hydroxypropylmethylcellulose, carboxymethylcellulose, sodium carboxymethylcellulose or mixtures thereof.

8. The controlled release delivery system for metformin according to claim 1 which comprises, the delivery system at least exhibiting a dissolution profile such that after about 2 hours, from about 12% to about 60% of the metformin is released; after about 4 hours, from about 25% to about 80% of the metformin is released; after about 8 hours, from about 50% to about 100% of the metformin is released; after about 12 hours, more than about 75% of the metformin is released.

9. A process for the preparation of controlled release delivery system for metformin which comprises,
a) granulating metformin with hydrophilic polymer(s) by using suitable solvent selected from water, isopropyl alcohol, ethyl alcohol, methylene chloride, acetone or mixtures thereof; the said hydrophilic polymer(s) is selected from group of polymers as claimed in claim 2,
b) optionally, using other pharmaceutical aids,
c) drying and sizing the obtained granules,
d) lubricating the granules of step(c) with hydrophobic lubricating agent(s) and compressing into a suitable dosage form; the said hydrophobic lubricating agent(s) is selected from the group of agents comprising pharmaceutically acceptable hydrophobic lubricating agent(s) on a weight basis, about 0.5% to about 15%, preferably present in an amount from about 1% to about 10%, more preferably from about 1.5% to about 7.5% by weight.

10. A process for the preparation of controlled release delivery system for metformin which comprises:
a) granulating metformin with hydrophilic polymer(s) by using suitable solvent selected from water, isopropyl alcohol, ethyl alcohol, methylene chloride, acetone or mixtures thereof; the said hydrophilic polymer(s) is selected from the group of polymers comprising hydroxypropyl methylcellulose, hydroxypropyl cellulose, sodium carboxymethylcellulose, carboxypolymethylene, carboxymethylcellulose calcium, carboxymethylcellulose sodium, hydroxyethyl cellulose, sodium alginate, xantham gum, or combinations thereof,
b) optionally, using other pharmaceutical aids,
c) drying and sizing the obtained granules,
d) further mixing the granules of step(c) with hydrophilic polymer(s) selected from said group of polymers,
e) lubricating the granules of step(d) with hydrophobic lubricating agent(s) and compressing into a suitable dosage form; the said hydrophobic lubricating agent(s) is selected from the group of agents comprising hydrogenated castor oil, hydrogenated cottonseed oil, hydrogenated palm oil, hydrogenated soybean oil, glyceryl monostearate, glyceryl diastearate, glycerol monooleate, glyceryl behenate, gylceryl palmitostearate, light mineral oil or combinations thereof.

11. The use of controlled release delivery system for Metformin in claim 1 for alleviating diseases caused by low insulin levels or insulin resistance as pathophysiological mechanism.

12. A method of treatment using a controlled release delivery system for Metformin as in claim 1 the medicament to a person in need thereof in pathophysiological conditions associated with low insulin levels or insulin resistance.
